(19) United States
(12) Patent Application Publication
(10) Pub. No.: US 2024/0268789 A1
(43) Pub. Date: Aug. 15, 2024

(54) INTEGRATED STEERABLE SHEATH ULTRASONIC IMAGING SYSTEM AND METHOD

(71) Applicant: SoundCath, Inc., Canoga Park, CA (US)

(72) Inventors: Donald Masters, Sylmar, CA (US); Jesus Andres Lopez, Bloomington, CA (US); Eric Stoppenhagen, Santa Monica, CA (US)

(73) Assignee: SoundCath, Inc., Canoga Park, CA (US)

(21) Appl. No.: 18/168,455

(22) Filed: Feb. 13, 2023

(51) Int. Cl. A61B 8/00; A61B 8/08; A61B 8/12

(52) U.S. Cl. CPC A61B 8/4494; A61B 8/0883; A61B 8/12; A61B 8/4254; A61B 8/445; A61B 2562/028

(57) ABSTRACT

An ultrasonic imaging system is disclosed. The ultrasonic imaging system comprises an Intracardiac echocardiographic (ICE) catheter and a transducer ring. A steerable sheath integrated with a built-in forward looking transducer and the transducer ring positioned at a distal end of the steerable sheath. The transducer ring comprises a MEMS based pMUT array arranged over a substrate. A catheter shaft houses a lumen to allow a passage of a puncture needle and an electronic flex cable in communication with at least one signal trace, configured to: direct the MEMS based pMUT array, via the at least one signal trace, to transmit and receive, ultrasound beams; receive at least one signal from the MEMS based pMUT array based on transmitting and receiving at least one ultrasound beam; and construct at least one image of at least a portion of the heart based on the at least one signal.

### FIELD OF THE DISCLOSURE

The present disclosure relates generally to the field of ultrasonic imaging systems. More particularly, some embodiments relate to a steerable sheath with an integrated a forward-looking intra-cardiac echocardiography (ICE) ultrasound catheter or a forward looking intra-cardiac echocardiography (ICE) ultrasound catheter with a lumen to allow the passage of a transseptal needle. The disclosed device would improve accuracy of a transseptal puncture and also facilitate the delivery of devices into the left atrium.

### BACKGROUND OF THE DISCLOSURE

Atrial fibrillation is the most common type of cardiac dysrhythmia that now affects approximately 2.2 million adults in the United States alone. Minimally-invasive catheter-based electrophysiological (EP) interventions provide valuable information about the electrical behaviour of the cardiac muscle that yields to better diagnosis and treatment of arrhythmias. catheter-based radio-frequency (RF) ablation, which is the most common ablation therapy, is often used to destroy a small amount of the malfunctioning tissue that causes the arrhythmia.

Use of catheter-based structural and electrophysiological procedures have recently expanded to more complex scenarios, in which an accurate definition of variable individual cardiac anatomy is a key to obtain optimal results. Intracardiac echocardiography (ICE) is a unique imaging modality for high-resolution real-time visualization of cardiac structures, continuous monitoring of catheter location within the heart, and early recognition of procedural complications, such as pericardial effusion or thrombus formation. Further, ICE imaging modality includes additional benefits, such as excellent patient tolerance, reduction of fluoroscopy time, and elimination of need for general anaesthesia or second operator.

Since its introduction, transseptal catheterization is used for left atrial access for the treatment of several conditions and is generally considered to be safe and effective. In the last years, there was an increasing number of different transcatheter interventions requiring this approach. The precision of the site of puncture is important not only to reduce the risk of complications but also to facilitate the delivery of devices into the desired portion of the left atrium and therefore the whole procedure. To facilitate transseptal catheterization, intracardiac echocardiography and transesophageal echocardiography (TEE) have been widely used to monitor the procedure and improve the safety and precision of puncture. Currently, ICE imaging modality has largely replaced trans-oesophageal echocardiography as ideal imaging modality for guiding certain procedures, such as atrial septal defect closure and catheter ablation of cardiac arrhythmias, and has an emerging role in others, including mitral valvuloplasty, transcatheter aortic valve replacement, and left atrial appendage closure.

In electrophysiology procedures, ICE imaging modality allows integration of real-time images with electro-anatomic maps. ICE imaging modality has a role in assessment of arrhythmogenic substrate and is particularly useful for mapping structures that are not visualized by fluoroscopy, such as the interatrial or interventricular septum, papillary muscles, and intracavitary muscular ridges. For these reasons, ICE has largely replaced trans-oesophageal echocardiography (TEE). Further, the introduction of ICE represents a major advancement in cardiac imaging and has become an integral part of a variety of percutaneous interventional and electrophysiology procedures, potentially improving outcomes and reducing risks. ICE allows a real-time assessment of cardiac anatomy during interventional procedures and guides catheter manipulation in relation to the different anatomic structures.

In contrast to TEE, ICE is performed by the primary operator of the interventional procedure under conscious sedation, without the need for endotracheal intubation, and thereby eliminate the risk of oesophageal trauma and other post anaesthesia outcomes. In addition, ICE reduces fluoroscopy exposure to both the patient and the operator, may improve outcomes, shortens the procedure time, and facilitates early recognition of complications such as thrombus formation or pericardial effusion.

Therefore, there is a need for an improved ultrasonic imaging system using a forward looking ultrasonic ICE catheter, with a new concept relating to improving the accuracy of the transseptal puncture.

### SUMMARY OF THE DISCLOSURE

By way of introduction, the preferred embodiments described below include an easy-to-use ultrasonic imaging system is disclosed. The ultrasonic imaging system comprises an Intracardiac echocardiography (ICE) catheter having a longitudinal axis, a proximal end, and a distal end. Further, a transducer ring positioned at the distal end of the ICE catheter. The transducer ring comprises a substrate and a micro-electromechanical (MEMS) based Piezoelectric Micro-Machined Ultrasonic Transducer (pMUT) array arranged over the substrate. The MEMS based pMUT array is a forward facing assembly. The MEMS based pMUT array comprises a plurality of pMUT array elements mounted on the substrate in a circular fashion or linear fashion. Further, the ultrasonic imaging system comprises a catheter shaft connected at one end to a handle assembly and at other end to the MEMS based pMUT array. The catheter shaft houses a lumen to allow a passage of a puncture needle and an electronic flex cable towards the proximal end of the ICE catheter. The electronic flex cable is in communication with at least one signal trace, and is configured to: direct each of the MEMS based pMUT array, via the at least one signal trace, to transmit and receive, with respect to heart, ultrasound beams having a bandwidth including a predetermined fundamental mode vibration of each of the plurality of pMUT array elements, such that a single array element can transmit and receive multiple fundamental mode vibrations simultaneously: receive at least one signal from the MEMS based pMUT array based on transmitting and receiving at least one ultrasound beam of the ultrasound beams, and construct at least one image of at least a portion of the heart based on the at least one signal. Further, the ultrasonic imaging system comprises a steerable sheath integrated with a built-in forward looking transducer and the transducer ring positioned at the distal end of the steerable sheath or the ICE catheter.

Further, the ICE catheter comprises a steering control unit positioned within the handle assembly, for articulating a distal tip of the ICE catheter and aligning face of the MEMS based pMUT array towards internal views including a fossa ovalis. The distal tip of the ICE catheter is coated with a material to provide electrical isolation and transmission of ultrasound signals. The ICE catheter corresponds to a mechanical flexible sheath with a marker band, to allow passage into the heart, and form a location on an X-ray image. Further, the ICE catheter is coupled to an imaging device using a custom dongle. The custom dongle is coupled to the handle assembly using an interposer and a flat circuit board. The custom dongle is configured to communicate ultrasound transmit pulses and ultrasound receive waveforms between the ICE catheter and the imaging device. Further, the catheter shaft encloses a plurality of individual electronic flex cables connected between the handle assembly and the MEMS based pMUT array. The ultrasound beams having a bandwidth including a predetermined fundamental mode vibration of each of a plurality of pMUT array elements, such that a single array element transmits and receives multiple fundamental mode vibrations simultaneously.

In one embodiment, an Intracardiac echocardiography (ICE) catheter is disclosed. The ICE catheter comprises a body having a longitudinal axis and a distal end. Further, a transducer ring positioned at the distal end of the ICE catheter. The transducer ring comprises a substrate and a micro-electromechanical (MEMS) based Piezoelectric Micro-Machined Ultrasonic Transducer (pMUT) array arranged over the substrate. The MEMS based pMUT array is a forward facing assembly. The MEMS based pMUT array comprises a plurality of transducer array elements arranged on the substrate. Further, the ICE catheter comprises a steerable sheath integrated with a built-in forward looking transducer and the transducer ring positioned at the distal end of the ICE catheter. Further, each of the plurality of transducer array elements comprises individual elements of multiple diameters. Further, the MEMS based pMUT array is connected in series between at least one signal trace and a common ground. Further, each transducer array element comprises a plurality of transducers, with a first group of two or more transducers in a first transducer array element and a second group of two or more transducers in the first transducer array element. Further, each of the plurality of transducer array elements are connected in parallel. Further, at least one first electrode is connected between the at least one piezoelectric layer and a signal conductor, and at least one second electrode is connected between the at least one piezoelectric layer and a ground conductor.

In one embodiment, an intracardiac echocardiographic (ICE) imaging system is disclosed. The ICE imaging system comprises an ICE catheter having a longitudinal axis, a proximal end, and a distal end. Further, a micro-electromechanical system (MEMS) based Piezoelectric Micromachined Ultrasonic Transducer (pMUT) array is disposed of within the distal end of the ICE catheter. The MEMS based pMUT array is a forward facing assembly and comprises a plurality of MEMS based pMUT array elements arranged on a substrate. Also, the MEMS based pMUT array comprises pMUT cells of multiple diameters to achieve a bandwidth of greater than 55%. Further, the ICE imaging system comprises a steerable sheath integrated with a built-in forward looking transducer and the transducer ring positioned at a distal end of the ICE catheter. Further, the ICE imaging system comprises a catheter shaft connected at one end to a handle assembly and at other end to the MEMS based pMUT array, and the catheter shaft houses a lumen to allow a passage of a puncture needle and an electronic flex cable towards the proximal end of the ICE catheter. The electronic flex cable is in communication with at least one signal trace and is configured to: direct each of the plurality of MEMS based pMUT array elements, via the at least one signal trace, to transmit and receive, with respect to heart, ultrasound beams; receive at least one signal from the plurality of MEMS based pMUT array elements based on transmitting and receiving at least one ultrasound beam of the ultrasound beams, and construct at least one image of at least a portion of the heart based on the at least one signal.

Other features and aspects of this disclosure will be apparent from the following description and the accompanying drawings.

### BRIEF DESCRIPTION OF THE DRAWINGS

The accompanying drawings illustrate various embodiments of systems, methods, and embodiments of various aspects of the disclosure. Any person of ordinary skill in the art will appreciate that the illustrated element boundaries (e.g., boxes, groups of boxes, or other shapes) in the figures represent one example of the various boundaries representative of the disclosed invention. It may be that in some examples one element may be designed as multiple elements or that multiple elements may be designed as one element. In other examples, an element shown as an internal component of one element may be implemented as an external component in another and vice versa. Furthermore, elements may not be drawn to scale. Non-limiting and non-exhaustive descriptions of the present disclosure are described with reference to the following drawings. The components in the figures are not necessarily to scale, emphasis instead being placed upon the illustrated principles.

Various embodiments will hereinafter be described in accordance with the appended drawings, which are provided to illustrate and not to limit the scope of the disclosure in any manner, wherein similar designations denote similar elements, and in which:

FIG. 1A is a schematic of a forward looking piezoelectric micro-machined ultrasonic transducer (pMUT) circular array assembly, according to an embodiment of the present disclosure:

FIG. 1B is a schematic of a forward looking pMUT linear array assembly, according to an embodiment of the present disclosure;

FIG. 2 illustrates a sectional view of a distal end of the ICE catheter with a plurality of transducer array elements, according to an embodiment of the present disclosure:

FIG. 3 illustrates a schematic diagram of an ultrasonic imaging system, according to an embodiment of the present disclosure;

FIGS. 4A and 4B illustrate a prior art imaging system, for acquiring two-dimensional image information;

FIG. 5 illustrates a perspective view of the distal end of the ICE catheter, according to an embodiment of the present disclosure;

FIG. 6 illustrates a cross-sectional view of a heart for placement of the forward looking ICE catheter before transsseptal puncture, according to an exemplary embodiment of the present disclosure;

FIG. 7 illustrates another cross-sectional view of the heart for the placement of the forward looking ICE catheter during the transsseptal puncture, according to an exemplary embodiment of the present disclosure;

FIG. 8 illustrates a schematic view of the ICE catheter, according to an embodiment of the present disclosure; and

FIG. 9 illustrates a multi-channel electronic communication between an ultrasonic imaging device and an MEMS based pMUT array, according to an embodiment of the present disclosure.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items or meant to be limited to only the listed item or items.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems, and methods are now described. The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

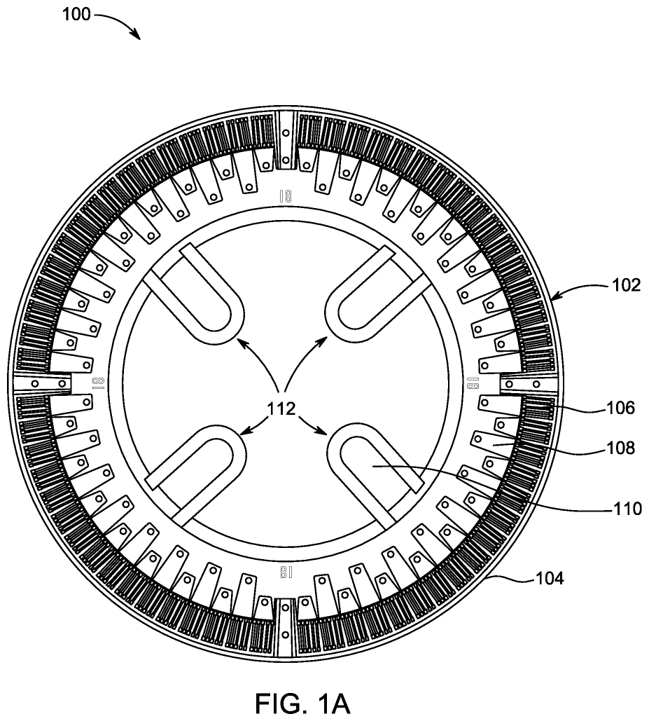

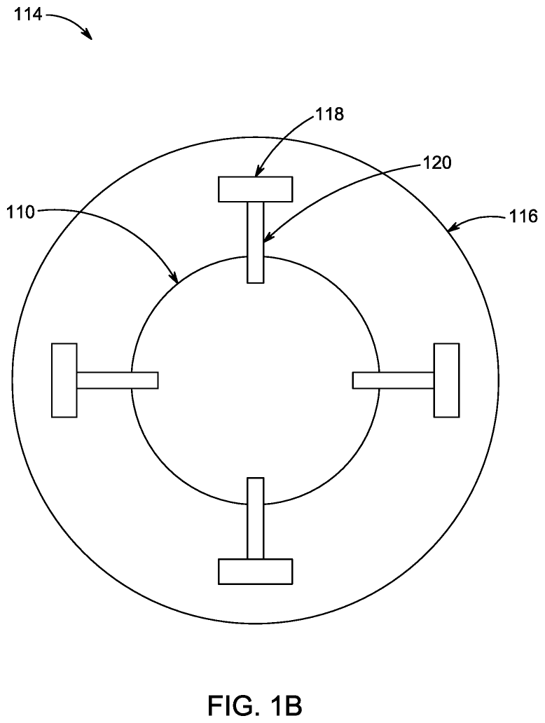

Referring to FIG. 1A, a schematic of a forward looking piezoelectric micro-machined ultrasonic transducer (pMUT) circular array assembly 100, is disclosed, according to an embodiment of the present disclosure.

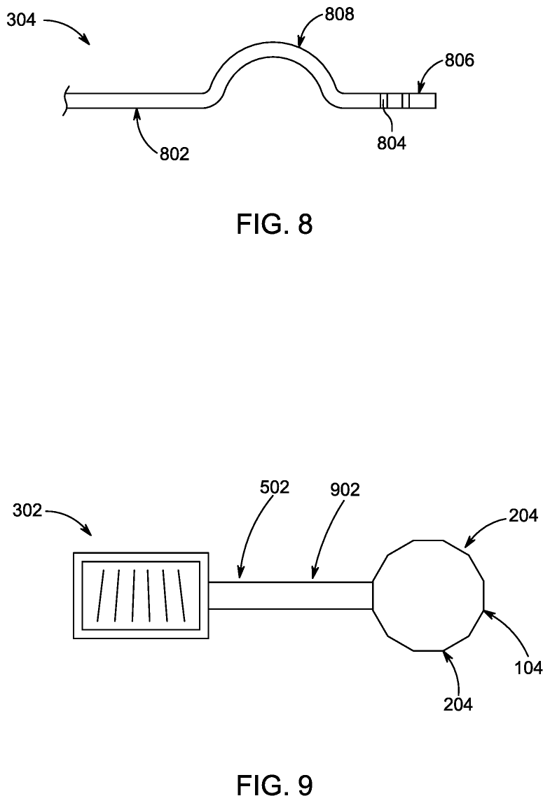

The pMUT circular array assembly 100 may be coupled to an Intracardiac echocardiography (ICE) catheter (not shown). The ICE catheter may have a longitudinal axis, a proximal end, and a distal end. The pMUT circular array assembly 100 may be positioned towards the distal end of the ICE catheter. The pMUT circular array assembly 100 may comprise to a circular transducer ring 102. Further, the circular transducer ring 102 may comprise a substrate 104 and a plurality of micro-electromechanical (MEMS) based pMUT array elements 106 mounted over the substrate 104 in a circular fashion. Further, the MEMS based pMUT array elements 106 is a forward facing assembly. Further, the substrate 104 may comprise a first plurality of connections 108 positioned along perimeter of the circular transducer ring 102. The first plurality of connections 108 may be configured to couple the MEMS based pMUT array elements 106 in multiple connections. It can be noted that the multiple connections may be series and/or parallel connections of the MEMS based pMUT array elements 106 with the substrate 104. Further, the first plurality of connections 108 positioned along perimeter of the circular transducer ring 102. Further, the MEMS based pMUT array elements connections 108 are routed through a lumen 110 via electronic flex cables 112. The circular transducer ring 102 may be positioned at the distal end of the ICE catheter. Further, the circular transducer ring 102 may be configured to transmit ultrasound beams forward of the distal end of the ICE catheter. The ICE catheter is described in conjunction with FIG. 8.

Referring to FIG. 1B, a schematic of a forward looking pMUT linear array assembly 114 is disclosed, according to an embodiment of the present disclosure.

The pMUT linear array assembly 114 may comprise to a linear transducer ring 116. The linear transducer ring 114 may comprise an MEMS based pMUT array elements 118 mounted over the substrate 104 in a linear fashion. The MEMS based pMUT array elements 118 may correspond to individual linear transducers. Further, the linear transducer ring 116 may comprise a second plurality of connections 120. Further, the MEMS based pMUT array elements 118 are routed through the lumen 110 via the electronic flex cables 112. Further, the linear transducer ring 116 may be positioned at the distal end of the ICE catheter and transmits ultrasound beams forward of the distal end of the ICE catheter.

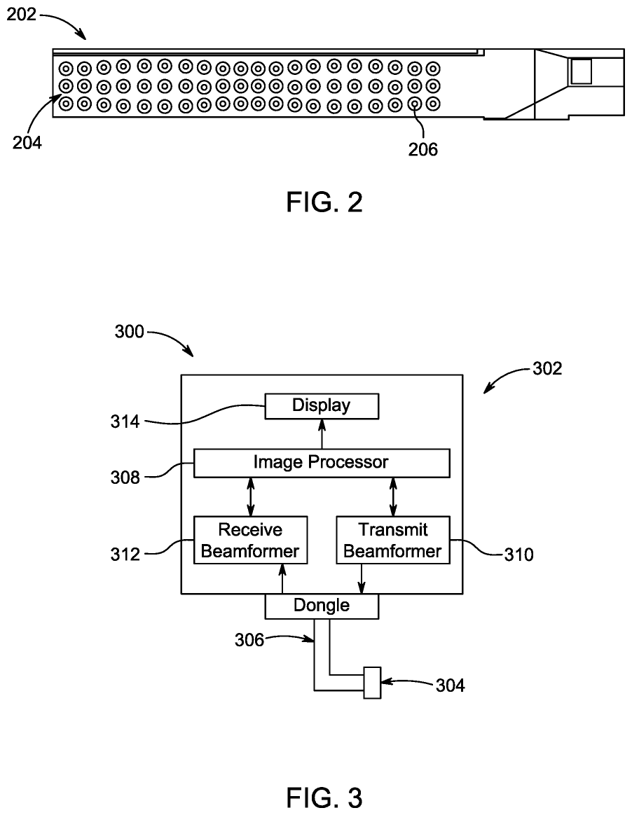

FIG. 2 illustrates a sectional view of a distal end of the ICE catheter with an MEMS based pMUT array 202 having a plurality of transducer array elements 204, according to an embodiment of the present disclosure.

The distal end of the ICE catheter may be provided with the MEMS based pMUT array 202 having the plurality of transducer array elements 204. Further, each of the plurality of transducer array elements 204 may have a plurality of individual transducer cells 206 arranged in a manner to provide a wide bandwidth of the individual focussed beam. In one embodiment, the MEMS based pMUT array 202 may be constructed from a pMUT array containing individual elements of different diameters. In one embodiment, to achieve wider bandwidth with pMUT arrays, multiple diameters of pMUT cells may be integrated into one element. It can be noted that by arranging pre-shaped pMUTs with different diameters, a broader bandwidth can be realized through the complex interaction between the individual pMUT elements. In one embodiment, the pMUT cells of multiple diameters may achieve a bandwidth of greater than 55%. For example, in 3 elements, there are 5 different dome diameters, and each array is of a different size, such as 300 μm.

Further, the MEMS based pMUT array 202 may correspond to pMUT and the plurality of transducer array elements 204 may correspond to a plurality of pMUT elements. In one embodiment, the plurality of pMUT elements may be directed to transmit and receive, the ultrasound beams having the bandwidth including the predetermined fundamental mode vibration of each of the plurality of pMUT elements, such that a single pMUT element can transmit and receive multiple fundamental mode vibrations simultaneously. In one embodiment, an electronic flex cable inside a catheter shaft of the ICE catheter receives the at least one signal from the plurality of pMUT elements. It can be noted that the at least one signal may correspond to the at least one ultrasound beam. The at least one signal may be transmitted to an ultrasonic imaging device 302, as shown in FIG. 3, for further processing in an image processor. The image processor may construct the at least one image of the heart. It can be noted that the plurality of pMUT elements may be used to create the individual focussed beam.

In one alternate embodiment, the MEMS based pMUT array 202 may include a cover portion that presents a flat cross-section. It can be noted that a feature of the MEMS based pMUT array 202 is typical in ultrasonic imaging catheters. Due to the severe space restrictions imposed by the small diameter of intracardiac catheters, the MEMS based pMUT array 202 is typically limited to a circular phased array made up of several individual transducer elements, such as 64 transducers or elements. The transducers have a flat surface from which sound is omitted and echoed sound is received. As is well known in the art, the individual transducer elements are pulsed by an ultrasound control system so that the emitted sound waves are constructively combined into a primary beam. By varying the time at which each transducer element is pulsed, an ultrasonic imaging system 300, as shown in FIG. 3, may render the individual beams into a focused image in order to obtain the 2D image. As a result, the MEMS based pMUT array 202 emits ultrasound along a plane that is perpendicular to the face of the transducer arrays. Thus, the MEMS based pMUT array 202 emits sound along a plane that is perpendicular to the assembly.

Referring to FIG. 3, a schematic diagram of the ultrasonic imaging system 300 is disclosed, according to an embodiment of the present disclosure.

The ultrasonic imaging system 300 may be performed for electrophysiology (EP). The ultrasonic imaging system 300 may be used for diagnosis and/or treatment in combination with another imaging modality, such as an x-ray, fluoroscopy, magnetic resonance, computed tomography, or optical system. Both imaging modalities may scan a patient for generating images to assist a physician. The data from the different modalities may be aligned by locating the markers with a known spatial relationship to the ultrasound scan in the images of the other modality. In other embodiments, the ultrasonic imaging system 300 may use a catheter without the markers and/or without another imaging modality. In one embodiment, the ultrasonic imaging system 300 may utilize a microelectromechanical (MEMS) transducer array defined as piezoelectric micro-machined ultrasound transducer (pMUT) or other types of MEMS transducers, interconnected using matched flexible circuits. In one embodiment, the ultrasonic imaging system 300 may correspond to an intracardiac echocardiographic (ICE) imaging system. In one embodiment, the ultrasonic imaging system 300 may correspond to an endovascular MEMS ultrasonic transducer utilizing a high-density flexible circuit for all transmission and electrical interconnects. In one embodiment, the ultrasonic imaging system 300 may be employed to treat patient with cystic fibrosis (CF). It can be noted that the use of the high-density flexible circuits may enable highly repeatable and stable transmission and return signals. Further, the high density flexible circuit transmission lines may transmit electrical energy from one end to another distal end of the ultrasonic imaging system 300.

The ultrasonic imaging system 300 may comprise the imaging device 302 coupled to an ICE catheter 304 via a communication channel 306. In one embodiment, the communication channel 306 may be a custom dongle with a cable and bus connections or multiple connections. Hereinafter, the communication channel 306 may be referred to as the custom dongle 306. In one embodiment, the ICE catheter 304 may correspond to an ultrasonic catheter.

The ICE catheter 304 may be disposed within a chamber of a heart of a patient and the imaging device 302 may receive at least one signal from the ICE catheter 304. The at least one signal may be communicated from the ICE catheter 304 to the imaging device 302 via the custom dongle 306. Further, the imaging device 302 may comprise an image processor 308, a transmit beamformer 310, a receive beamformer 312, and a display 314.

The image processor 308 may be configured to generate a two-dimensional (2D) image according to data received from the ICE catheter 304. In one embodiment, the image processor 308 may be configured to receive a focussed signal from the receive beamformer 312. The image processor 308 may render the data to construct an image or sequence of images. In one embodiment, the image may be three dimensional (3D) representation, such as a two-dimensional image rendered from a user or a processor selected viewing direction. In one embodiment, the image processor 308 may be a detector, filter, processor, application-specific integrated circuit, field-programmable gate array, digital signal processor, control processor, scan converter, three-dimensional image processor, graphics processing unit, analog circuit, digital circuit, or combinations thereof. The image processor 308 may receive beamformed data and may generate images, to display on the display 314. It can be noted that the generated images are associated with a two-dimensional (2D) scan. Alternatively, the generated images may be three-dimensional (3D) representations.

The image processor 308 may be programmed for hardware accelerated two-dimensional re-constructions. The image processor 308 may store processed data of the at least one signal and a sequence of images in a memory. In one embodiment, the memory may be a non-transitory computer-readable storage media. The instructions for implementing the processes, methods and/or techniques discussed herein are provided on the computer-readable storage media or memories, such as a cache, buffer, RAM, removable media, hard drive, or other computer-readable storage media. Non-transitory computer-readable storage media include various types of volatile and non-volatile storage media. The functions, acts, or tasks illustrated in the figures or described herein are executed in response to one or more sets of instructions stored in or on a computer readable storage media. The functions, acts, or tasks are independent of the particular type of instructions set, storage media, processor, or processing strategy and may be performed by software, hardware, integrated circuits, firmware, micro code, and the like, operating alone or in combination.

The transmit beamformer 310 may be configured for transmission of the electrical signal or electrical impulse in a form of at least one signal towards the ICE catheter 304. The receive beamformer 312 may be configured to receive an electrical signal or electrical impulse from the ICE catheter 304. In one embodiment, the transmit beamformer 310 and the receive beamformer 312 may facilitate transmit beamforming technique to focus energy towards a receiver to improve a signal to noise (SNR) of the at least one signal and then transmit the at least one signal to the image processor 308.

The display 314 may be configured to screen the image or sequence of images during or after the data is rendered, by the image processor 308. The image may be three dimensional (3D) representation, such as a two-dimensional image rendered from a user or a processor selected viewing direction. Alternatively, the image may be one or more two-dimensional images representing planes in the volume. In one embodiment, the display 314 may be a part of imaging device 302 or may be remote, such as a networked display. In one embodiment, the display 314 may be a cathode ray tube (CRT), liquid crystal display (LCD), a projector, a plasma, or other now known or later developed display device.

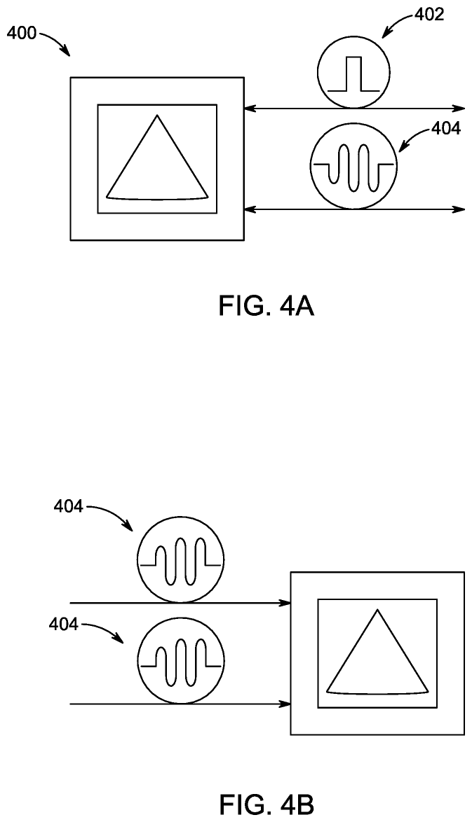

Referring to FIGS. 4A and 4B, a prior art imaging system 400 is disclosed. The imaging system 400 may be used for diagnosis and/or treatment in combination with another imaging modality, such as an x-ray, fluoroscopy, magnetic resonance, computed tomography, or optical system. It can be noted that imaging modalities scan a patient for generating images to assist a physician. Further, the imaging system 400 provides an ultrasound transmit pulse 402 and an ultrasound receive path 404, for connection to an ultrasonic transducer (not shown). The ultrasound transmit pulse 402 may transmit ultrasound signals from the imaging system 400 towards an object such as heart of a patient. Further, the ultrasound receive path 404 may create a waveform based at least one of the ultrasound signals. Thereafter, the imaging system 400 may convert the received ultrasound signals or ultrasound information to a two-dimensional (2D) image of the object or a portion of the object.

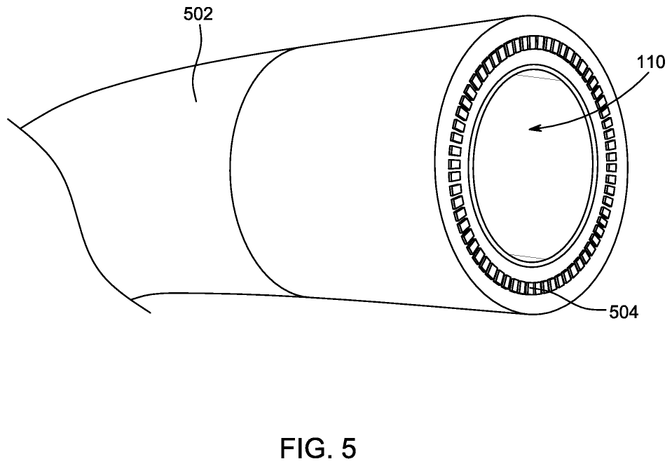

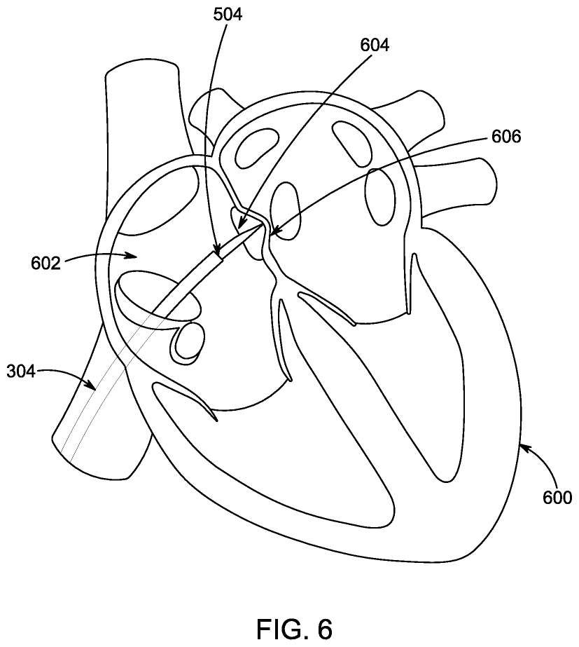

Referring to FIG. 5, a perspective view of the distal end of the ICE catheter 304 is disclosed, according to an embodiment of the present disclosure.

The ICE catheter 304 may comprise a catheter shaft 502 housing the lumen 110. The lumen 110 may allow passage of a puncture needle (not shown) and a flex cable (not shown). It can be noted that the flex cable communicates ultrasound signals between a transducer array 504 and the dongle 306. The transducer array 504 may include the MEMS based pMUT array elements 106 arranged along the periphery of the circular transducer ring 102.

Referring to FIG. 6, a cross-sectional image of a heart 600 with placement of the forward looking ICE catheter 304 before the transsseptal puncture according to an exemplary embodiment of the present disclosure.

The ICE catheter 304 may be positioned within a right atrium 602 of the heart 600. Further, the ICE catheter 304 may comprise a distal tip 604. The distal tip 604 of the ICE catheter 304 may be inserted into the right atrium 602 via an inferior vena cava (not shown). The movement of the distal tip 604 of the ICE catheter 304 within the right atrium 602 may be controlled by a steering control unit (not shown) of the ICE catheter 304 to position for imaging a fossa ovalis 606.

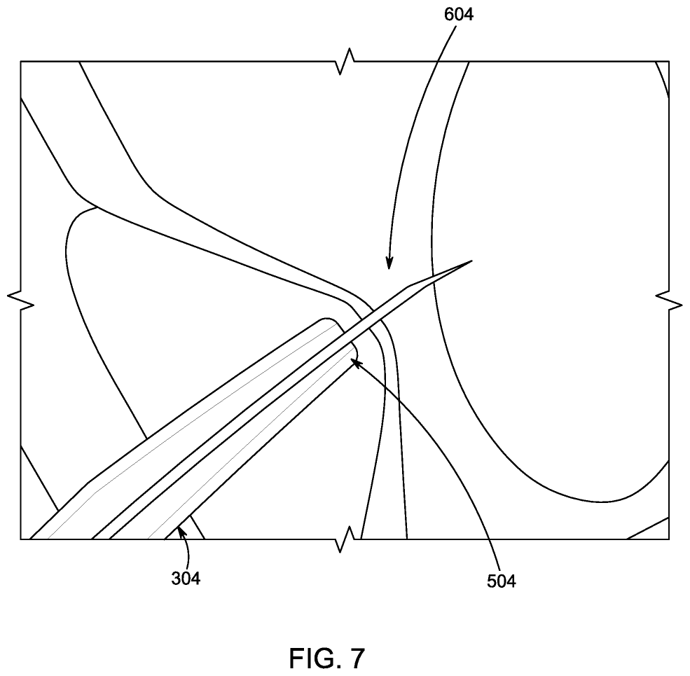

Referring to FIG. 7, another cross-sectional view of the heart 600 for the placement of the forward looking ICE catheter 304 during the transsseptal puncture is disclosed, according to an exemplary embodiment of the present disclosure.

The distal tip 604 of the ICE catheter 304 may be positioned within the right atrium 602 of the heart 600. The steering control unit may be actuated to advance the distal tip 604 of the ICE catheter 304 to puncture the fossa ovalis 606.

Referring to FIG. 8, a schematic view of the ICE catheter 304 is disclosed, according to an embodiment of the present disclosure.

The ICE catheter 304 may comprise a flexible sheath 802 with a marker band 804 to allow location on an X-ray image (not shown). The flexible sheath 802 may have the marker band 804 towards a distal end 806 of the ICE catheter 304, to allow a passage into the chamber of the heart 600 of the patient and thereby allow location on the X-ray image. It can be noted that the distal end 806 of the ICE catheter 304 may be coated with a material to provide electrical isolation and transmission of ultrasonic signals generated by the ICE catheter 304. In one embodiment, the flexible sheath 802 may be inserted inside the chamber of the heart 600 and the marker band 804 may allow location on the X-ray image. It can be noted that the image processor 308 of the ultrasonic imaging device 302 may provide a real-time 2D image of the heart using the allowed location on the X-ray image. In one embodiment, the flexible sheath 802 may correspond to the catheter shaft 304 to allow the passage into the heart and thereby achieve location on the X-ray image. In one embodiment, the patient's having CF may be treated with the ICE catheter 304 coated with electrical isolation for transmission of ultrasonic signals generated by the ICE catheter 304. In one embodiment, the flexible sheath 802 may correspond to a steerable sheath integrated with a built-in forward looking transducer and the transducer ring 102 positioned at the distal end 806 of the steerable sheath or the ICE catheter 304. It can be noted that the steerable sheath with an integrated the forward-looking ICE catheter 304 or a forward looking ICE catheter with the lumen 110 may facilitate a passage for the puncture needle or transseptal needle. The steerable sheath may facilitate maximum maneuvering of the ICE catheter 304 to allow deflection of the puncture needle. It can also be noted that the steerable sheath may facilitate access to hard-to-reach areas inside the heart.

Further, the ICE catheter 304 may comprise an electrically isolated shaft 808 towards the distal end 806 of the ICE catheter 304. The electrically isolated shaft 808 may use a copolymer material up to the distal end 806 of the ICE catheter 304. In one embodiment, the electrically isolated shaft 808 may be coated with Pebax material. The imaging window may allow ultrasound beams to pass back and forth to the MEMS based pMUT array 202. Further, the distal tip 806 of the ICE catheter 304 is coated with an electrically isolated material to provide isolation and transmission of the ultrasound signals.

Further, the MEMS based pMUT array 202 may be disposed within the distal end 806 of the ICE catheter 304. The MEMS based pMUT array 202 may comprise the plurality of transducer array elements 204 arranged on the substrate 104. Further, the MEMS based pMUT array 202 may be connected in series between at least one signal trace and a common ground. Further, each of the plurality of transducer array elements 204 may comprise a plurality of transducers, with a first group of two or more transducers in a first transducer array element and a second group of two or more transducers in the first transducer array element. Further, each of the plurality of transducer array elements 204 may be connected in parallel. Further, each transducer array element may comprise at least one piezoelectric layer disposed on the substrate 104. It can be noted that the at least one piezoelectric layer may comprise the pMUT array element. Further, each transducer array element may comprise at least one first electrode connected between the at least one piezoelectric layer and a signal conductor. Further, at least one-second electrode may be connected between the at least one piezoelectric layer and a ground conductor. In one embodiment, each pMUT array element may have a predetermined geometry configured to accept a predetermined fundamental mode vibration.

In one embodiment, the MEMS based pMUT array 202 may comprise a plurality of pMUTs coupled at the distal end 806 of the ICE catheter 304. It can be noted that the pMUT array is a circular phased array. In one embodiment, the first group of two or more transducers and the second group of two or more transducers may be connected in parallel. Further, the multiple transducer array elements of the plurality of transducer array elements may be grouped to act as a single array element.

Referring to FIG. 9, a multi-channel electronic communication between the ultrasonic imaging device 302 and the MEMS based pMUT array 202, according to an embodiment of the present disclosure.

The MEMS based pMUT array 202 may comprise the plurality of transducer array elements 204 arranged on the substrate 104. Further, each of the plurality of transducer array elements 204 may provide a wide bandwidth of an individual focussed beam. The MEMS based pMUT array 202 may be coupled to the ultrasonic imaging device 302 using a dongle cable. The MEMS based pMUT array 202 disposed within the distal end 806 of the ICE catheter 304 may transmit the at least one signal via an electronic flex cable 902 inside the catheter shaft 502 to the ultrasonic imaging device 302. The at least one signal may be the acoustic echo transmitted from the MEMS based pMUT array 202. It can be noted that the acoustic echo of acoustic energy may be received from a face of the MEMS based pMUT array 202 and received at the image processor 308.

Further, the ultrasound beams may have a bandwidth including a predetermined fundamental mode vibration of each of the plurality of transducer array elements 204, such that a single array element can transmit and receive multiple fundamental mode vibrations simultaneously. It can be noted that the plurality of transducer array elements 204 may transmit and receive the ultrasound beams with respect to the heart or at least a portion of the heart. Further, the electronic flex cable 902 inside the catheter shaft 502 may be configured to receive at least one signal from the plurality of transducer array elements 204 based on transmitting and receiving at least one ultrasound beam of the ultrasound beams. The ultrasonic imaging device 302 may be further configured to construct at least one image of at least the portion of the heart based on the at least one signal. It can be noted that the electronic flex cable may be configured to the transmit beamformer 310 and receive beamformer 312 to display a two-dimensional (2D) image information of the heart or the at least portion of the heart.

In one embodiment, the plurality of transducer array elements 204 may correspond to a micro-electromechanical (MEMS) based Piezoelectric Micromachined Ultrasonic Transducers (pMUTs). The catheter shaft 502 may be connected to a handle assembly (not shown) at one end and to the MEMS based pMUT array 204 at other end. The electronic flex cable 902 inside the catheter shaft 502 may be in communication with the at least one signal trace. It can be noted that the electronic flex cable 902 may be further communicate to the transmit beamformer 310 and the receive beamformer 312, via the custom dongle 306 to display a two-dimensional (2D) image information of the heart to be scanned.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. An ultrasonic imaging system comprising:
an Intracardiac echocardiography (ICE) catheter having a longitudinal axis, a proximal end, and a distal end;
a transducer ring positioned at the distal end of the ICE catheter, wherein the transducer ring comprises a substrate and a micro-electromechanical (MEMS) based Piezoelectric Micro-Machined Ultrasonic Transducer (pMUT) array arranged over the substrate, wherein the MEMS based pMUT array is a forward facing assembly, and comprises a plurality of pMUT array elements mounted on the substrate in a circular fashion or linear fashion;
a steerable sheath integrated with a built-in forward looking transducer and the transducer ring positioned at a distal end of the ICE catheter;
a catheter shaft connected at one end to a handle assembly and at other end to the MEMS based pMUT array, wherein the catheter shaft houses a lumen to allow a passage of a puncture needle and an electronic flex cable towards the proximal end of the ICE catheter, the electronic flex cable is in communication with at least one signal trace, and is configured to:
direct the MEMS based pMUT array, via the at least one signal trace, to transmit and receive, with respect to heart, ultrasound beams;
receive at least one signal from the MEMS based pMUT array based on transmitting and receiving at least one ultrasound beam of the ultrasound beams; and
construct at least one image of at least a portion of the heart based on the at least one signal.

2. The ultrasonic imaging system of claim 1, wherein each of the plurality of pMUT array elements having transducer cells of multiple diameters, to achieve a wide bandwidth.

3. The ultrasonic imaging system of claim 1, wherein the transducer ring is configured to transmit ultrasound beams forward of the distal end of the ICE catheter.

4. The ultrasonic imaging system of claim 1, wherein the transducer ring corresponds to a circular transducer ring with the MEMS based pMUT array mounted over the substrate in a circular fashion.

5. The ultrasonic imaging system of claim 1, wherein the transducer ring corresponds to a linear transducer ring with the MEMS based pMUT array mounted over the substrate in a linear fashion.

6. The ultrasonic imaging system of claim 1, wherein the ICE catheter comprises a steering control unit positioned within the handle assembly, for articulating a distal tip of the ICE catheter and aligning face of the MEMS based pMUT array towards internal views including a fossa ovalis.

7. The ultrasonic imaging system of claim 6, wherein the distal tip of the ICE catheter is coated with a material to provide electrical isolation and transmission of ultrasound signals.

8. The ultrasonic imaging system of claim 1, wherein the ICE catheter corresponds to a mechanical flexible sheath with a marker band, to allow passage into the heart, and form a location on an X-ray image.

9. The ultrasonic imaging system of claim 1, wherein the ICE catheter is coupled to an imaging device using a custom dongle, and the custom dongle is configured to communicate ultrasound transmit pulses and ultrasound receive waveforms between the imaging device and the ICE catheter.

10. The ultrasonic imaging system of claim 1, wherein the catheter shaft encloses a plurality of individual electronic flex cables connected between the handle assembly and the MEMS based pMUT array.

11. The ultrasonic imaging system of claim 1, wherein the ultrasound beams having a bandwidth including a predetermined fundamental mode vibration of each of a plurality of pMUT array elements, such that a single array element transmits and receives multiple fundamental mode vibrations simultaneously.

12. An Intracardiac echocardiography (ICE) catheter comprising:
a body with a longitudinal axis and a distal end;
a transducer ring positioned at the distal end of the ICE catheter, wherein the transducer ring comprises a substrate and a micro-electromechanical (MEMS) based Piezoelectric Micro-Machined Ultrasonic Transducer (pMUT) array arranged over the substrate, wherein the MEMS based pMUT array is a forward facing assembly, and comprises a plurality of transducer array elements arranged on the substrate;
a steerable sheath integrated with a built-in forward looking transducer and the transducer ring positioned at the distal end of the ICE catheter,
wherein each transducer array element comprises a plurality of transducers, with a first group of two or more transducers in a first transducer array element and a second group of two or more transducers in the first transducer array element, and each transducer array element is connected in parallel, and comprising:
at least one piezoelectric layer disposed on the substrate;
at least one first electrode connected between the at least one piezoelectric layer and a signal conductor; and
at least one second electrode connected between the at least one piezoelectric layer and a ground conductor.

13. The ICE catheter of claim 12, wherein each of the plurality of transducer array elements is a linear phased array.

14. The ICE catheter of claim 12, wherein each of the plurality of transducer array elements is a circular phased array.

15. The ICE catheter of claim 12, wherein the plurality of transducer array elements creates an individual focused beam.

16. The ICE catheter of claim 12, further comprising an electrically isolated shaft to cover a shaft up to an imaging window at the distal end of the body.

17. The ICE catheter of claim 16, wherein the electrically isolated shaft uses Pebax material to cover the shaft up to the imaging assembly at the distal end of the body.

18. The ICE catheter of claim 12, wherein the ICE catheter comprises a steering control unit for articulating a distal tip of the ICE catheter and aligning face of the MEMS based pMUT array towards internal views including a fossa ovalis.

19. The ICE catheter of claim 18, wherein the distal tip of the ICE catheter is coated with a material to provide electrical isolation and transmission of ultrasound signals.

20. An intracardiac echocardiographic (ICE) imaging system comprising:
an ICE catheter having a longitudinal axis, a proximal end, and a distal end;
a micro-electromechanical (MEMS) based Piezoelectric Micromachined Ultrasonic Transducer (pMUT) array disposed within the distal end of the ICE catheter, wherein the MEMS based pMUT array is a forward facing assembly and comprises a substrate and a plurality of MEMS based pMUT array elements arranged on the substrate, and pMUT cells of multiple diameters to achieve a bandwidth of greater than 55%;
a steerable sheath integrated with a built-in forward looking transducer and a transducer ring positioned at a distal end of the ICE catheter; and
a catheter shaft connected at one end to a handle assembly and at other end to the MEMS based pMUT array, and the catheter shaft houses a lumen to allow a passage of a puncture needle and an electronic flex cable towards the proximal end of the ICE catheter, wherein the electronic flex cable is in communication with at least one signal trace, and is configured to:
direct each of the plurality of MEMS based pMUT array elements, via the at least one signal trace, to transmit and receive, with respect to heart, ultrasound beams;
receive at least one signal from the plurality of MEMS based pMUT array elements based on transmitting and receiving at least one ultrasound beam of the ultrasound beams; and
construct at least one image of at least a portion of the heart based on the at least one signal.

21. The ICE imaging system of claim 20, wherein each of the plurality of MEMS based pMUT array elements having transducer cells of multiple diameters, to achieve a wide bandwidth.

22. The ICE imaging system of claim 20, wherein the MEMS based pMUT array corresponds to a transducer ring with the plurality of MEMS based pMUT array elements configured to transmit ultrasound beams forward of the distal end of the ICE catheter.

23. The ICE imaging system of claim 22, wherein the transducer ring corresponds to a circular transducer ring with the MEMS based pMUT array mounted over the substrate in a circular fashion.

24. The ICE imaging system of claim 22, wherein the transducer ring corresponds to a linear transducer ring with the MEMS based pMUT array mounted over the substrate in a linear fashion.
